(19) United States
(12) Patent Application Publication
(10) Pub. No.: US 2025/0041564 A1
(43) Pub. Date: Feb. 6, 2025

(54) MEDICAL DEVICE FOR THE TARGETED APPLICATION OF A THERAPEUTIC AGENT TO THE ROUND WINDOW OF THE COCHLEA

(71) Applicants: UNIVERSITE DE BORDEAUX, BORDEAUX (FR); INSTITUT NATIONAL DE LA SANTE ET DE LA RECHERCHE MEDICALE – INSERM, PARIS (FR); CENTRE HOSPITALIER UNIVERSITAIRE DE BORDEAUX, TALENCE CEDEX (FR); INSTITUT PASTEUR, PARIS (FR)

(72) Inventors: Damien BONNARD, PESSAC (FR); Raphaël DEVILLARD, BORDEAUX (FR); Didier DULON, CESTAS (FR); Olivia KEROUREDAN, MERIGNAC (FR)

(21) Appl. No.: 18/713,519

(22) PCT Filed: Nov. 28, 2022
(86) PCT No.: PCT/EP2022/083469

§ 371 Date: May 24, 2024

(30) Foreign Application Priority Data
Nov. 29, 2021 (FR) FR2112660

(51) Int. Cl. A61M 25/00

(52) U.S. Cl. CPC A61M 25/0082; A61M 25/0023; A61M 25/005

(57) ABSTRACT

A medical device for the delivery of a therapeutic agent by bioprinting including a rod body including a bioprinting cartridge arranged near the distal end of the rod body including a top layer including a solution containing a therapeutic agent; and an absorbent compound capable of converting light energy from laser radiation into thermal energy and arranged to cause heating of the solution including a therapeutic agent to cause a jet of the solution; an optical fiber extending longitudinally within a lumen of the rod body to deliver laser flow onto the absorbing compound.

### FIELD OF THE INVENTION

The invention relates to a medical device for the targeted application of a therapeutic agent. In particular, the invention relates to a medical device for the targeted application of a therapeutic agent to small, difficult-to-access areas, such as the round window of the cochlea. The invention is particularly advantageous in the treatment of disorders of the neurosensory epithelium of the inner ear.

### STATE OF THE ART

The cochlea is the coiled part of the inner ear containing the auditory nerve endings, and is the site of many ear disorders.

Numerous therapeutic agents in development or already available, such as gene therapy agents, aim to treat various disorders of the human inner ear, responsible for deafness, tinnitus or vertigo. To inject an agent to treat these conditions, the round window is the preferred route of entry. In adult humans, the round window has a diameter of around 2 mm. The round window comprises a 40-60 μm-thick membrane made up of 3 cell layers. This membrane vibrates in opposition to the acoustic vibrations entering the inner ear. It acts as a pressure valve, allowing waves to travel through the cochlea's fluids. This membrane provides privileged access to the fluids in the cochlea, which is surrounded by bone.

A known method for injecting such agents into the inner ear is direct intracochlear injection via the round window using a microcatheter. The catheter pierces and then passes through the round window to inject the therapeutic agent.

One disadvantage is the high risk of neurosensory lesion and consequent sequelae of deafness following microperforation of the round window membrane (mechanical trauma to cochlear structures). For these reasons, this method is not applicable to humans in current clinical practice.

A local transtympanic injection method is also known. The therapeutic agent is injected (in liquid or gel form) in large quantities into the middle ear. This method relies on natural transmembrane diffusion mechanisms. As a result, a quantity of agent in contact with the round window will diffuse and migrate inside the cochlea.

One disadvantage of this method is that it allows no control over the penetration of the therapeutic agent into the inner ear, and therefore over the dose administered. Another drawback is the resulting quantity of product in the middle ear, which can cause contamination of surrounding tissues. Indeed, it is very difficult to deposit such a substance very precisely on the round window. For example, the humidity in the middle ear makes such precision difficult. What's more, the middle ear canal is connected to the nasal passages. With such a method, there is therefore a significant risk of inhalation of the therapeutic agent by the subject.

As a result, there is a need for injecting these substances into the inner ear that simultaneously allow good control of the dose delivered, absence of contamination of surrounding tissues and atraumaticity on the sensory structures of the inner ear.

The present invention proposes to solve this problem by overcoming the disadvantages mentioned.

### SUMMARY OF THE INVENTION

According to one aspect, the invention relates to a medical device for the delivery of a therapeutic agent by bioprinting comprising a rod body. The rod body comprises a bioprinting cartridge arranged near the distal end of the rod body. Said cartridge comprises a top layer comprising a solution comprising a therapeutic agent and comprises an absorbent compound capable of converting light energy from laser radiation into thermal energy and arranged to cause heating of said solution comprising a therapeutic agent to cause a jet of said solution. The medical device further comprises an optical fiber extending longitudinally within a lumen of the rod body for directing laser flow onto the absorbent compound.

The advantage of using bioprinting is that it enables droplets of a few picoliters in size to be generated, containing the therapeutic agent, at high speed and in a highly targeted manner on the round window. Indeed, the thermal energy converted by the absorbent compound is transmitted to the solution in the upper layer, creating local vaporization of said solution until the formation of a vapor bubble whose explosion generates the ejection of a microdroplet comprising the therapeutic agent. A further advantage is that the therapeutic agent is thus applied to the round membrane without direct contact therewith. The therapeutic agent thus applied will then diffuse into the fluids of the inner ear. The therapeutic agent can thus impact precisely on the surface of the round window membrane in an atraumatic way, without risk of lesions, and the dosage of the therapeutic agent is controlled.

In one embodiment, the cartridge further comprises an absorbent layer, comprising a lower surface and an upper surface being arranged against the upper layer; said absorbent layer comprising said absorbent compound.

One advantage is to concentrate the absorbent compound in contact with the top layer, promoting the transfer of thermal energy to the solution comprising the therapeutic agent.

In another embodiment, the absorbent compound is diluted in the top layer.

One advantage is to facilitate the manufacture of the cartridge and to reduce the volume of said cartridge. Another advantage is to convert the light energy of the beam into thermal energy directly at the heart of the solution comprising the therapeutic agent, thus facilitating vapor bubble generation and droplet ejection comprising the therapeutic agent.

In one embodiment, the bioprinting cartridge further comprises a transparent support plate, arranged to support the top layer.

One advantage is to physically support the top layer, which may be liquid or viscous, while allowing the light beam to pass through with minimum energy loss.

In one embodiment, the absorbent layer is a metal coating on the upper surface of the support plate.

In one embodiment, the support plate is a glass plate. The glass is advantageously transparent to ultraviolet and infrared wavelengths.

In one embodiment, the absorber compound comprises a metal such as gold, titanium or silver. These compounds are particularly advantageous for generating thermal energy from a light beam.

In one embodiment, the thickness of the absorbent layer is between 10 nm and 150 nm.

In one embodiment, the therapeutic agent comprises a therapeutic agent for intra-cochlear treatment.

In one embodiment, the therapeutic agent is in liquid form.

In one embodiment, the solution comprising the therapeutic agent comprises a liquid solution, a viscous solution or a gelled solution into which the therapeutic agent is diluted.

In one embodiment, the medical device further comprises a focusing lens arranged between the cartridge and the distal end of the optical fiber to focus the laser beam exiting the optical fiber onto the absorbent compound.

In one embodiment, the medical device further comprises rod body guide means.

In one embodiment, the cartridge is arranged in a receptacle of the rod body, optionally near the distal end of the rod body.

In one embodiment, the cartridge is removably arranged in the receptacle. One advantage is that the rod can be reused by replacing the cartridge.

In one embodiment, the medical device further comprises connection means, at the proximal end of the optical fiber, for connecting the optical fiber to a laser light source.

In one embodiment, the shaft body is a catheter body.

In one embodiment, the medical device comprises a plurality of co-axial optical fibers each extending longitudinally within a lumen of the rod body to deliver a laser flow onto the absorbing compound at a different position.

According to a second aspect, the invention concerns a medical system comprising a medical device according to the invention and a laser source connected to the proximal end of the optical fiber.

In one embodiment, the laser source is designed to emit a pulsed laser beam. In one embodiment, the medical system further comprises means for directing a laser beam from the laser source to feed at least one of the medical device's co-axial optical fibers.

In one embodiment, the means for directing the laser beam are configured to feed the co-axial optical fibers one after the other, preferably sequentially. One advantage is that a microdrop can be dispensed one after the other at different positions on the cartridge, in order to accelerate the rate of deposition on the target and better distribute the therapeutic agent over the target surface.

### BRIEF DESCRIPTION OF FIGURES

Further features and advantages of the invention will become apparent from the following detailed description, with reference to the appended figures, which illustrate:

FIG. 1: a schematic view of the medical device according to the invention and of a system comprising said medical device and including means for generating a laser flow on a bioprinting cartridge at the distal end of the catheter according to one embodiment of the invention.

FIG. 2A: a schematic cross-sectional view of the bioprinting cartridge subjected to a laser flow causing a jet of a drop comprising a therapeutic agent according to one embodiment of the invention in which the cartridge comprises an absorbent layer.

FIG. 2B: a schematic cross-sectional view of the bioprinting cartridge subjected to a laser flow causing a jet of a drop comprising a therapeutic agent according to one embodiment of the invention in which an absorbent compound is diluted in the top layer.

FIG. 3: a schematic view of the distal end of the catheter including the end of the optical fiber and the passage of a laser flow through a focusing lens onto the bioprinting cartridge and a receptacle for receiving a cartridge according to one embodiment of the invention.

FIG. 4: a schematic view of the distal end of the catheter in which the cartridge is arranged on a side wall of the catheter body.

FIG. 5: A schematic view of the distal end of the catheter comprising distal walls forming a central passage for the passage of therapeutic agent projections. The distal walls are designed to be placed against the target, thus controlling the distance between the cartridge and the target.

FIG. 6: a schematic view of a cross-sectional plane of the medical device abutting the cochlea to project a therapeutic agent onto the round window membrane of the cochlea.

FIG. 7: a schematic view of the medical device according to an embodiment of the invention and of a system comprising said medical device and including means for generating a laser flow on a bioprinting cartridge at the distal end of the catheter according to an embodiment of the invention in which the catheter comprises a plurality of co-axial optical fibers. A control element is arranged to divert the laser flow to one of the co-axial fibers according to instructions supplied by a computer.

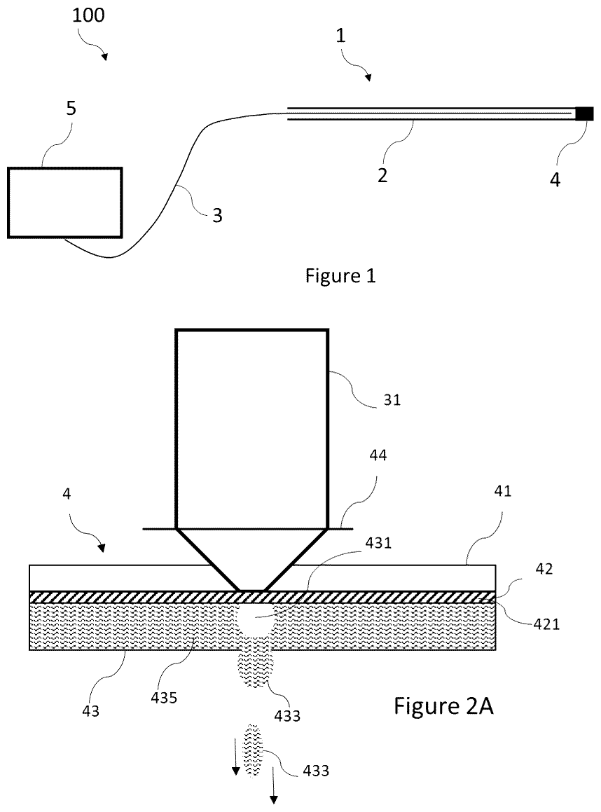

### DESCRIPTION OF THE INVENTION

The invention consists of a medical device comprising a shaft such as a catheter enabling the projection onto a membrane target (such as the round window) of a bio-ink comprising a therapeutic agent.

The bio-ink is arranged on a cartridge comprising an absorbent compound capable of transferring the light energy of a laser beam into thermal energy. The heat generated by the absorbent compound in the bio-ink vaporizes the bio-ink locally, generating a vapor bubble in the depth of the bio-ink, which expands and collapses to form a bio-ink jet.

Surprisingly, the inventors discovered that such a jet of bio-ink on the round window membrane enables very precise targeting of the round window, as well as improved transmembrane transfer of the therapeutic agent, without damaging the round window membrane or contaminating surrounding tissues or spaces, while guaranteeing good control of the therapeutic agent dosage.

### ROD

The medical device 1 according to the invention comprises a rod.

The rod must be understood as an elongated body, the distal part of which is designed to be inserted into a canal or orifice.

The shaft body is preferably a catheter body 2.

In the remainder of this description, the term “catheter body” will be used to designate such a shaft body.

The catheter body 2 extends longitudinally from a proximal end intended for handling by the operator to a distal end 21.

The distal end 21 of the catheter body is preferably designed to be inserted into a subject's middle ear via a minimally invasive approach known from otological surgery. In a preferred example, the catheter body is designed to be inserted through an incision in the walls near the eardrum.

The cross-section of the catheter body is preferably circular, ovoid, oval or any shape without a straight edge. One advantage is to reduce the risk of tissue damage when inserting the catheter body into a channel or orifice.

The catheter body is preferably rigid, enabling better control of the catheter body's position and orientation during the bioprinting procedure.

The catheter may comprise means for guiding a distal portion of the catheter body. The guiding means are configured to orient the distal end 21 in a predetermined direction, in particular relative to the direction of the longitudinal axis of the proximal portion of the catheter body. One advantage is that it is easier to aim at the round window once the catheter tip has been inserted into the middle ear.

The catheter body 2 may comprise one or more lumens extending inside the catheter body and running along the longitudinal axis of the catheter body.

In particular, the distal end 21 of the catheter body 2 is designed to be introduced into a subject's middle ear, for example via the eardrum or via a bore in the bone wall of the subject's skull.

The distal end 21 of the catheter body 2 is preferably of the same order of magnitude as the round window of a subject. For example, the dimensions of the catheter body according to a cross-sectional plane perpendicular to the longitudinal axis of said body 2 are between 0.1 mm and 5 mm, preferably between 1 mm and 3 mm.

### Optical Fiber

The medical device comprises one or more optical fibers 3. The optical fiber 3 is designed to carry laser flow from the proximal end of the catheter body to a distal part of the catheter body 2.

The optical fiber 3 is arranged within a lumen of the catheter body 2.

Preferably, the medical device 1 comprises a connector for connecting the optical fiber 3 to a laser source 5 so as to route a laser flow from the laser source 5 to the distal end 32 of the optical fiber.

In an embodiment illustrated in FIG. 3, optical fiber 3 extends to face the bioprinting cartridge 4. The distal end 32 of the optical fiber 3 and the bioprinting cartridge 4 are arranged so that the laser beam 31 is directed towards the bioprinting cartridge 4.

In one embodiment, the distal end 32 of the optical fiber 3 is arranged at a distance of 5 cm or less from the bioprinting cartridge 4. In a particular embodiment, the distal end 32 of the optical fiber 3 is arranged in contact with the cartridge 4. To this end, the optical fiber may comprise means for generating, at its distal end, a focused light beam. In an alternative embodiment, the distal end 32 of the optical fiber is arranged at a distance from the bioprinting cartridge 4 of between 1000 and 3000 μm.

In one embodiment, the bioprinting cartridge 4 is arranged in line with the longitudinal axis of the optical fiber.

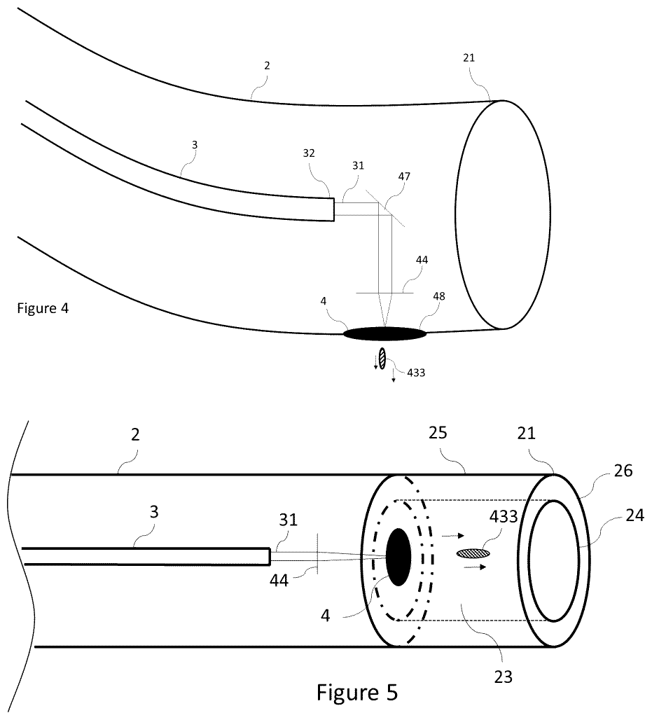

The medical device 1 may also include optical means for guiding the laser beam 31 towards the bioprinting cartridge 4. In an example shown in FIG. 4, the medical device 1 comprises at least one mirror 47 arranged and oriented to deflect the laser beam 31 towards the bioprinting cartridge 4.

In one embodiment, the bioprinting cartridge 4 is arranged to project a jet of bioink through a side window 22 of the catheter body 2. The mirror 47 then deflects the laser beam 31 in the direction of the lower surface of the bioprinting cartridge 4.

In one embodiment, the distal end of the laser fiber is movable relative to the cartridge so as to be able to illuminate the cartridge at different successive points. In another embodiment, optical means, such as optical lenses and/or mirrors, are designed to illuminate the cartridge at different successive points, for example to scan the cartridge with the laser beam.

This makes it possible to create projections from several points on the top layer, and thus project a scan of the target.

### Multiple Optical Fibers

Several optical fibers can be placed coaxially to multiply the points of impact on the absorbent layer or on the cartridge 4. The plurality of optical fibers can be arranged in the same lumen or each in a coaxial lumen of the catheter body.

In an embodiment illustrated in FIGS. 7 and 8, the medical device 1 comprises a plurality of coaxial optical fibers.

The coaxial optical fibers are for conveying a laser stream into one or more lumens of the catheter body. The coaxial optical fibers are arranged from the proximal end of the catheter body to a distal part of the catheter body 2.

Each optical fiber is arranged so that a laser beam from the distal part of the optical fiber reaches the cartridge 4 at a different point. One advantage of the plurality of coaxial laser fibers is that they enable the cartridge to be scanned without having to mechanically move the cartridge, the fiber, the catheter body, or a lens located between the distal part of the optical fiber and the cartridge. In fact, depending on the optical fiber fed by a laser flow, it is possible to illuminate a different zone of the cartridge.

Preferably, the device comprises at least 9 coaxial optical fibers to illuminate at least 9 zones of the cartridge.

In an embodiment illustrated in FIG. 8, at least one of the laser fibers is designed to illuminate the target with light (preferably laser light) in the visible range. One advantage of this is that it enables the operator to illuminate the target and facilitate guidance of the distal part of the catheter. To this end, the medical device further comprises a second laser connected to power the optical fiber for illuminating the target.

Said fiber can be arranged to illuminate the target through the cartridge. In one particular mode, the cartridge may comprise a through hole to allow the light beam emitted by said fiber to pass through. In another mode, the cartridge is sufficiently transparent to ensure illumination of the target by the light beam passing through the thickness of the cartridge.

### Cartridge

The bioprinting cartridge 4 (also referred to as “cartridge” in this description) comprises at least one absorbent compound and a top layer comprising the therapeutic agent.

By “upper”, we mean the direction towards the target or the direction opposite to the direction of origin of the laser beam on cartridge 4. By “lower”, we mean the direction from which the laser beam 31 arrives on the cartridge 4.

By “bioprinting”, we mean the deposition of a substance (called “bioink” in this description) comprising a therapeutic agent on living tissue, by projecting a jet of this substance.

### Top Layer

The top layer 43 comprises a therapeutic agent. Preferably, the therapeutic agent comprises an inner ear treatment agent and/or a gene therapy agent. Preferably, top layer 43 comprises a therapeutic agent dispersed or diluted in a liquid phase or in a viscous or gelled phase, such as a phase comprising a hydrogel.

In one particular mode, the therapeutic agent is diluted in a phase designed to be in gel form at least at body temperature (around 36° C.). One advantage is to improve absorption of the therapeutic agent through the round window, as the gel-like deposit guarantees mechanical stability of the drop expelled onto the membrane. In a first example, said phase is designed to be liquid at room temperature (about 23° C.) to improve cartridge manufacture. In a second example, the therapeutic agent is diluted in a phase designed to be in gelled form at body and room temperature. The fact that the top layer is gelled advantageously enables the cartridge to be handled without the risk of losing the top layer, and also makes it possible to manufacture larger cartridge sizes.

The liquid phase preferably comprises an aqueous liquid. The liquid phase may comprise serum such as fetal calf serum. This liquid phase comprising the therapeutic agent is referred to below as “bio-ink”.

The therapeutic agent can be in liquid or solid form, for example in the form of a medicated powder.

In one embodiment, the therapeutic agent comprises fluorescent latex beads, cells, liposomes (containing plasmids, Adeno-Associated-Virus) and/or pharmacological substances.

The therapeutic agent may include a marking agent.

The top layer 43 extends in a plane of predefined thickness. The thickness of top layer 43 can be between 100 μm and 10 mm, more preferably between 100 μm and 5 mm.

Cartridge diameter can range from 100 μm to 3 mm.

Preferably, the volume of the top layer is between 15 and 100 μL.

The top layer may comprise a fluorescent agent as a marking agent, such as fluorescein. In an alternative or cumulative embodiment, the top layer comprises a corticoid substance such as dexamethasone.

To enable a drop comprising the therapeutic agent to be sprayed, the top layer 43 is arranged opposite an outlet of the catheter body 2, for example its distal end 21 or a side window 22.

The top layer 43 of cartridge 4 can be arranged so that its plane is oriented substantially perpendicular to the longitudinal axis of the catheter body. This arrangement advantageously allows droplets 433 comprising the therapeutic agent to be projected in a direction substantially parallel to the longitudinal axis of the catheter body. In this case, the target, for example the round window, is advantageously easier for the operator to aim at.

### Absorbent Compound

The cartridge comprises an absorbent compound. The absorbent compound comprises at least one compound capable of converting light energy from the laser beam into thermal energy.

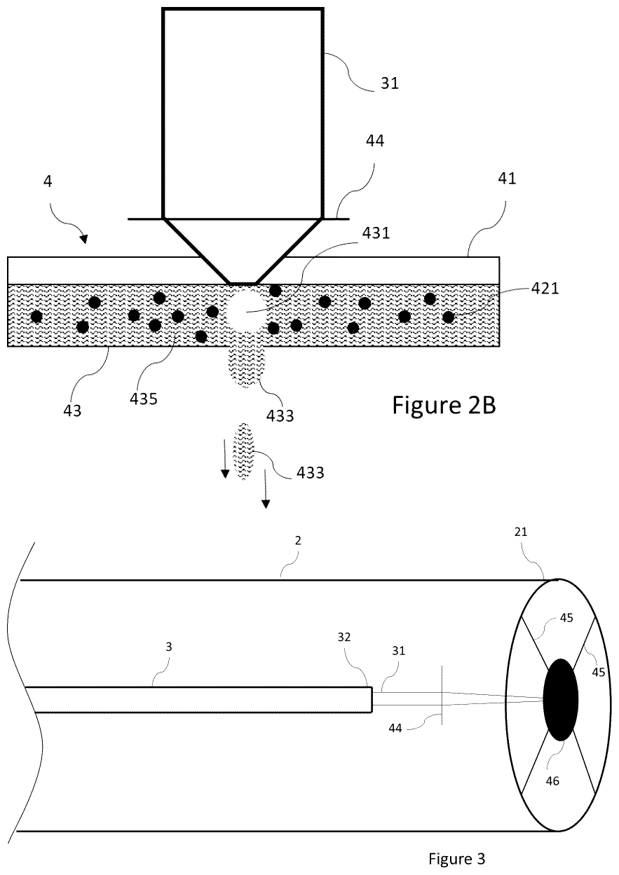

In a first embodiment illustrated in FIG. 2A, the cartridge 4 comprises an absorbent layer 42. Absorbent layer 42 is a thin layer comprising at least one compound 421 capable of converting light energy from the laser beam into thermal energy. Preferably, absorber layer 42 is a metal layer.

As it heats up, the absorbent layer 42 will transmit at least some of this thermal energy to the top layer. Heating the liquid contained in this upper layer 43 will generate a bubble 431 in contact with the absorbent layer 42. In effect, the bio-ink liquid, in contact with the absorbent layer 42, is heated until it causes local vaporization in contact with the absorbent layer 42. Heating leads to the growth of a gas bubble 431 in contact with the absorbent layer 42.

The expansion of the gas bubble 431 and its collapse, after reaching a critical size, induce the formation of a bio-ink jet 433 comprising the therapeutic agent. As a result, a microdrop 433 of bio-ink is transferred from the top layer in a direction substantially perpendicular to the plane of the top layer.

Laser beam 31 supplies the energy required to create and eject a picoliter-sized droplet at high speed, which is then projected onto a target substrate.

Preferably, the absorber layer 42 comprises a metal compound such as gold, platinum or silver. These metals have the advantage of being good thermal conductors and of efficiently transferring light energy into thermal energy.

The absorbent layer 42 may comprise a metallic layer such as gold, platinum or silver.

Alternatively, the absorbent layer 42 may comprise a polymer layer formulated to improve absorbency.

The thickness of the absorber layer 42 is preferably between 10 and 150 nm. The advantage of such a thickness is to reduce the distance between its lower surface receiving the laser beam and its upper surface in contact with the upper layer. Such an absorbent layer 42 is therefore more efficient at transferring heat to the upper layer.

Preferably, the absorbent layer 42 is a coating layer.

In a second embodiment illustrated in FIG. 2B, the same objective can be achieved by diluting the solution 435 in the top layer 43 with said absorbent compound 421. For example, metallic particles such as gold, platinum or silver particles can be dispersed in the top layer. Metallic particles dispersed in the solution 435 of the top layer advantageously reduce the volume of the cartridge and facilitate its manufacture. In this case, the cartridge 4 comprises only the support layer 41 and the top layer 43 superimposed on the support layer 41. Another advantage is that it is easier to generate “droplet” ejection, since heat is generated directly in the volume of the top layer rather than on its surface.

In this embodiment, the absorbent compound 421 and the therapeutic agent are diluted in a liquid or gel matrix. This matrix is preferably transparent to the laser radiation emitted by the distal end of the optical fiber.

Preferably, the concentration and thickness of the absorbing compound 421 in the top layer are set so that 50 to 100% of the laser light energy is absorbed by the absorbing compound 421 in said top layer 43.

### Support Plate

In one embodiment, cartridge 4 further comprises a support plate 41. The support plate 41 preferably comprises a rigid material for supporting the absorbent layer 42 and/or the top layer 43.

In one embodiment, the absorbent layer 42 is arranged between a support plate 41 and the top layer 43 comprising the therapeutic agent. The absorbent layer 42 is preferably a coating deposited on the top surface of the support plate 41 in contact with the top layer 43. In this case, the support plate 41 must be transparent to the laser beam 31 to enable the laser beam 31 to reach the absorbent layer 42.

The support plate 41 is preferably in contact with the absorbent layer 42 so that the absorbent layer 42 is sandwiched between the support plate 41 and the top layer comprising the therapeutic agent.

In another embodiment shown in FIG. 2B, the support plate 41 is arranged in contact with the top layer 43.

In general, the support layer is arranged to support the top layer. The thickness of the support plate 41 is preferably between 200 μm and 5 mm.

The bioprinting cartridge 4 is arranged so that the laser beam 31 emanating from the distal end 32 of the optical fiber 3 reaches the lower surface of the absorbent layer 42 or the absorbent compound 421, optionally through the support plate 41.

Preferably, support layer 41 is a glass plate. Glass is particularly advantageous for its transparency properties. The laser beam can thus reach the absorbing compound through the support plate, particularly at infrared or ultraviolet wavelengths.

### Laser Beam Focusing

As illustrated in FIGS. 2A to 4, the catheter 1 further comprises one or more focusing lenses 44 (also known as converging lenses). The focusing lens 44 enables the laser beam 31 emanating from the distal end 32 of the optical fiber to be focused on the absorbent layer 42 or on the absorbent compound 421 diluted in the solution 435 containing the therapeutic agent, advantageously enabling light energy to be concentrated in the solution 435 and thus more rapidly generate a bubble 431 in the upper layer and a bio-ink drop jet.

The focusing lens 44 is preferably arranged at a distance from the lower surface of the absorbent layer 42 equal to or substantially equal to its focal length. In another mode, this focusing lens 44 is arranged at a distance from the upper layer 43 equal to or substantially equal to its focal length.

The focal length is the distance between the geometric center of the lens and the point (focus) where a set of rays converge, parallel to each other, after passing through the focusing lens 44.

In this way, the absorbing compound 421, optionally the lower surface of the absorbing layer, is arranged at the focal point of the focusing lens 44, and the parallel rays arriving on the lens all converge on the same point of the absorbing compound 421. The thermal energy generated at this point is increased by the conversion of light energy into thermal energy. This facilitates the production of a bio-ink droplet jet.

In one embodiment, the focusing lens 44 is arranged in direct contact with the distal end of the optical fiber. For example, the focusing lens may be formed by the distal end of the optical fiber.

### Robotic Support

In one embodiment, the medical device 1 comprises means for controlling the translational and/or rotational movement of the catheter body.

Control means may include robotic means. One advantage is that the round window can be aimed more easily, and the catheter stabilized throughout the procedure.

The guiding means can be connected to optical means such as a camera or a laser sighting device. Advantageously, the optical means make it possible to maintain the orientation of the distal end 21 of the catheter relative to the subject. For example, the camera or laser sight is configured to detect movement of the subject and is configured to automatically generate a command to the control means to modify the orientation and/or position of the distal end as a function of the user's movement.

### Receptacle

In one embodiment, the bio-ink cartridge 4 is removable from the catheter. This advantageously enables a cartridge 4 to be replaced after use, and the remainder of the medical device 1 and catheter body 2 to be reused.

As such, the catheter body 2 comprises a receptacle 46 as illustrated in FIG. 3. The receptacle 46 is designed to receive the bio-ink cartridge 4. In one embodiment, the receptacle 46 is located at or near the distal end 21 of the catheter. Receptacle 46 is designed to receive bioprinting cartridge 4 from its lateral sides, leaving a free passage for laser beam 31 to the absorbent compound and leaving the bioprinting projection free in the direction substantially perpendicular to the plane of the top layer.

In one embodiment, illustrated in FIG. 3, the receptacle can be held by fixing arms 45 at the center of the catheter body section.

Receptacle 46 may comprise reversible connection means complementary to cartridge connection means to enable removable attachment between cartridge 4 and receptacle 46.

### Stop Support

In a first embodiment shown in FIG. 3, the cartridge is arranged at the distal end of the catheter.

In a second alternative embodiment shown in FIG. 5, the catheter body comprises a stop wall 25. The stop wall 25 extends longitudinally in the distal direction of the catheter body to a distal opening 24. The stop wall 25 forms a projection zone 24 between the cartridge and the distal end, allowing bio-ink drops 433 to pass from the cartridge to the distal opening 24.

The stop wall is designed to abut the target. The abutment of the distal end 26 of this wall 25 against the target advantageously ensures that the distance between the cartridge 4 and the target is maintained at a predetermined distance.

The stop walls 25 are side walls around the projection area 23. As illustrated in FIG. 5, the side walls may form a round, circular or hollow ovoid section around the passage area 23. The medical device may comprise a single tubular abutment wall 25 as shown in FIG. 5. The medical device may comprise several stop walls 25 of identical length around the projection area 23.

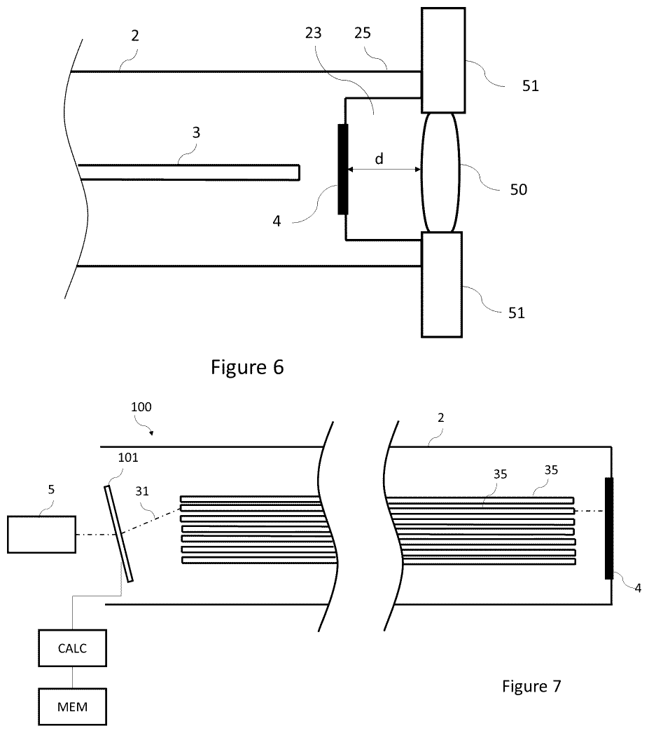

In an example shown in FIG. 6, the abutment walls 25 are placed against the cochlea bone 51 surrounding the cochlea round window membrane 50. A first advantage is to maintain the distance d between the cartridge 4 and the membrane 50. A second advantage is to provide support to stabilize the catheter head relative to the target, in this case the round window membrane 50.

Preferably, the stop wall(s) 25 are designed to create a distance d between the cartridge and the distal opening 26 of between 500 μm and 10,000 μm, very preferably between 1,000 μm and 5,000 μm.

In one embodiment, the medical device comprises means for monitoring the distance d between the cartridge and the distal end 26 of the stop walls 25.

To this end, the stop wall(s) 25 can be translationally movable relative to the cartridge 4. For example, the stop wall(s) 25 can be mounted on the catheter body on a screw thread to adjust the distance d between the cartridge 4 and the distal opening 26. In another example, the cartridge can be moved in translation to adjust the distance d between cartridge 4 and distal opening 26. In the latter example, cartridge 4 is integral with the optical fiber and/or integral with the optical means (focusing lens, mirrors) to ensure convergence of the laser beam at the absorbent compound 421.

Preferably, the distal ends of abutment walls 25 comprise a sensor for detecting that said abutment walls are in direct contact with cochlea bone 51. Said sensor may comprise a pressure sensor or an impedance sensor for detecting direct contact with the cochlea bone.

In a further alternative embodiment, the catheter body comprises at least two targeting optical fibers. The two aiming optical fibers are designed to emit a laser beam in the visible range. The two targeting optical fibers are arranged to emit laser beams that will intersect at a predetermined distance from the cartridge.

One advantage is that the user, when approaching the target with the catheter body, will visualize the two points of the two aiming laser beams. When the target is at a distance from the cartridge equal to the predetermined distance, the two points will merge and the user will visualize a single point.

Preferably, the targeted optical fibers are arranged so that their beams intersect at a distance d from the cartridge of between 500 μm and 10,000 μm.

### Medical System

The invention also relates to a medical system 100 comprising a medical device 1 as previously described and a laser source 5. The laser source 5 is designed to generate a laser beam.

Preferably, laser source 5 is designed to generate a laser beam with a wavelength of between 400 nm and 2 microns. The wavelength is advantageously selected in a wavelength in which the absorbent compound 421 is capable of transferring light energy into thermal energy. The laser source 5 is connected to the medical device 1 in such a way as to cause a laser flow generated by the laser source to pass through the optical fiber 3 of the medical device 1.

Any type of laser can be used. The laser source is preferably designed to emit a laser beam whose wavelength avoids damaging biological tissue.

Preferably, the laser source is designed to emit an infrared laser beam. Preferably, a laser source for emitting a laser beam with a wavelength between 1000 and 1100 nm.

The advantage of infrared light is that it reduces the risk of damaging biological tissue.

In one embodiment, the laser source is a semiconductor material. In particular, a laser source comprising an Nd-YAG (neodymium-doped yttrium aluminum garnet) laser can be used.

In other embodiments, ultraviolet light can be used.

Preferably, the laser source is designed to emit a pulsed laser beam. One advantage of pulsed light is that it enables bubble generation and microdroplet expulsion without a layer of absorbing compound.

In an embodiment where the medical device 1 comprises a plurality of coaxial optical fibers as described above, the medical system 100 may comprise a plurality of laser sources 5 and the proximal portion of each fiber is connected to a laser source 5. In another embodiment illustrated in FIG. 7, the medical system 100 comprises a laser source 5 and includes means 101 for directing the laser beam from the laser source 5 into a selected laser fiber. The means 101 for directing the laser beam 31 may comprise a mirror and/or at least one controllable optical lens for deflecting the laser beam towards the selected optical fiber.

In one embodiment, the controllable element is slaved to a controller such as a processor or a CALC computer.

In another embodiment, the medical system 100 comprises a first laser source 5 for causing a microdrop to be transferred to the target and a second light source for illuminating the target via an optical fiber as previously described.

In one embodiment, the system comprises means for acquiring images of a region in the longitudinal extension of the catheter body. Preferably, at least one of the coaxial optical fibers is connected to an image acquisition device. The system is then configured to acquire and optionally display in real time a video channel showing the target in front of the distal end of the catheter body during the approach.

### Illumination Sequence

When a microdrop is ejected from the top layer, a disturbance of the surface state is observed. After ejection, the top layer relaxes until it returns to a substantially flat surface. The time between the ejection of the microdrop and the return to this state is called the “relaxation time” and depends on the therapeutic agent and the phase in which said therapeutic agent is diluted in the upper layer.

When a microdrop is ejected when the surface state of the top layer is disturbed, the direction of the jet is more random and less precise.

In one embodiment, the medical system is configured to deflect light through the co-axial optical fibers described above in a predetermined sequence.

Preferably, the system is configured to leave a time between two shots from the same optical fiber equal to or greater than a predetermined time period. Said predetermined time period is preferably greater than the relaxation time of the top layer.

In one example, the predetermined sequence comprises the illumination of the cartridge by the plurality of optical fibers in a sequence in which two successive illuminations are never emitted by two adjacent optical fibers.

A first optical fiber is considered “adjacent” to a second optical fiber when the second optical fiber, among the plurality of co-axial optical fibers, is the one having the shortest distance to the first optical fiber or one of the three shortest distances to the first optical fiber.

This improves the accuracy of solution jetting with such a sequence.

The invention relates to a computer program product configured for the medical system to implement said sequence. The invention also relates to a computer-readable medium such as a MEM memory (preferably of the non-transitory type) comprising said computer program.

1. A medical device for delivery of a therapeutic agent by bioprinting comprising a rod body comprising:
a bioprinting cartridge arranged near a distal end of the rod body comprising:
a top layer comprising a solution containing a therapeutic agent; and
an absorbent compound capable of converting light energy from laser radiation into thermal energy and arranged to cause heating of said solution comprising a therapeutic agent to cause a jet of said solution;
an optical fiber extending longitudinally within a lumen of the rod body to deliver laser flow onto the absorbent compound.

2. The medical device according to claim 1, wherein the bioprinting cartridge further comprises an absorbent layer comprising said absorbent compound, said absorbent layer comprises a lower surface and an upper surface being arranged against the upper layer.

3. The medical device according to claim 1, wherein the absorbent compound is diluted in the top layer.

4. The medical device according to claim 1, wherein the bioprinting cartridge further comprises a transparent support plate, arranged to support the top layer.

5. The medical device according to claim 1, wherein the absorbent compound comprises a metal.

6. The medical device according to claim 1, wherein the therapeutic agent comprises a therapeutic agent for intra-cochlear treatment.

7. The medical device according to claim 1, wherein the solution comprising the therapeutic agent comprises a liquid solution, a viscous solution or a gelled solution in which the therapeutic agent is diluted.

8. The medical device according to claim 1, further comprising a focusing lens arranged between the bioprinting cartridge and the distal end of the optical fiber to focus the laser beam exiting from the optical fiber onto the absorbent compound.

9. The medical device according to claim 1, wherein the bioprinting cartridge is removably arranged in a receptacle of the rod body.

10. The medical device according to claim 1, comprising a plurality of co-axial optical fibers each extending longitudinally within a lumen of the rod body for directing a laser flow onto the absorbent compound at a different position.

11. The medical system comprising a medical device according to claim 1 and a laser source connected to the proximal end of the optical fiber.

12. A medical system, comprising:
a medical device according to claim 11,
a laser source,
means for directing the laser beam to feed the co-axial optical fibers one after the other.

13. The medical device according to claim 5, wherein the metal is gold, titanium or silver.
